(19) United States
(12) Patent Application Publication
(10) Pub. No.: US 2024/0242845 A1
(43) Pub. Date: Jul. 18, 2024

(54) METHODS AND MODLES FOR IDENTIFYING BREAST LESIONS

(71) Applicants: National Central University, Taoyuan City (TW); MacKay Memorial Hospital, Taipei City (TW)

(72) Inventors: Jia-Ching WANG, Taoyuan City (TW); Yi-Chiung HSU, Taoyuan City (TW); Bach-Tung PHAM, Taoyuan City (TW); Phuong-Thi LE, Taoyuan City (TW); Po-Sheng YANG, Taipei City (TW)

(21) Appl. No.: 18/411,061

(22) Filed: Jan. 12, 2024

(30) Foreign Application Priority Data
Jan. 13, 2023 (TW) 112101588

(51) Int. Cl. G16H 50/50; G06T 7/00; G06T 7/11; G06V 10/26; G06V 10/774; G06V 10/82

(52) U.S. Cl. CPC G16H 50/50; G06T 7/0012; G06T 7/11; G06V 10/273; G06V 10/774; G06V 10/82; G06T 2207/10116; G06T 2207/20021; G06T 2207/20081; G06T 2207/20084; G06T 2207/20221; G06T 2207/30068; G06T 2207/30096

(57) ABSTRACT

A method is provided for building a model to determine breast lesions in a subject. The method involves sequential process of image processing, segmentation, object detection, and masking on obtained mammographic images to obtain local images and extracted feature information of breast lesions. Following this, classification and training are conducted using the local images and feature information to establish the model. Also provided herein is a method for diagnosing and treating breast cancer with the aid of the model.

### CROSS-REFERENCES TO RELATED APPLICATIONS

This application claims priority and the benefit of Taiwan Patent Application No. 112101588, filed Jan. 13, 2023, the entirety of which is incorporated herein by reference.

### BACKGROUND OF THE INVENTION

### 1. Field of The Invention

The present disclosure relates to the field of diagnosis and treatment of breast cancers. More particularly, the disclosed invention relates to methods for determining and identifying a breast lesion of a subject based on his/her mammographic images, and treating the subject based on the identified breast lesion.

### 2. Description of Related Art

According to statistical data from the American Cancer Society (ACS), women over the age of 40 have a significantly high probability of developing breast cancer. This highlights the global concern among women regarding the prevention and early detection of breast cancer.

In current medical technology, breast imaging established through mammography (i.e., an X-ray imaging system for breasts) is predominantly utilized as a method for early prevention and detection of breast cancer. The diagnostic process for mammography adheres to the standards of the Breast Imaging Reporting and Data System (BI-RADS), which encompasses seven categories (0-6). Initially, the assessment involves determining the proportion occupied by fibroglandular tissue in the mammographic image to assess whether a patient belongs to a high-risk group for breast cancer and then the presence of a lesion in the mammographic image is confirmed. If a lesion is identified, further classification of the shape and margins of the lesion is conducted to determine its benign or malignant nature.

However, the above procedures for interpreting mammographic images are often performed by healthcare professionals through manual assessment, resulting in a significant time investment in the diagnostic process. Moreover, the manual interpretation of mammographic images relies on experience and subjective perception, leading to varying judgment criteria and outcomes among different healthcare professionals. This gives rise to challenges related to increased personnel and time costs, as well as issues regarding efficiency and accuracy.

In view of the foregoing, there exists in the related art a need for an improved method and system that can determine breast lesions in individual breast mammographic images.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, the purpose of the present disclosure is to provide a diagnostic model and method for identifying a breast lesion in a subject with the aid of mammographic images, such that the efficiency and accuracy in diagnosis of breast cancers can be highly improved.

In one aspect, the present disclosure is directed to a method for building a model for determining a breast lesion in a subject via mammographic images. The method comprises: (a) obtaining a plurality of mammographic images of the breast from the subject, in which each of the mammographic images comprises an attribute of the breast lesion; (b) producing a plurality of processed images via subjecting each of the plurality of mammographic images to image treatments, which comprise image cropping, image denoising, image flipping, histogram equalization, image padding, and a combination thereof; (c) segmenting each of the plurality of processed images of step (b) to produce a plurality of segmented images, and/or detecting the attribute on each of the plurality of processed images of step (b) to produce a plurality of extracted sub-images; (d) segmenting each of the plurality of extracted sub-images of step (c) to produce a plurality of segmented sub-images; (e) combining each of the extracted sub-images of step (c) and each of the segmented sub-images of step (d), thereby producing a plurality of combined images respectively exhibiting the attribute for each of the mammographic images; and (f) classifying and training the plurality of combined images of step (e) with the aid of a convolutional neural network, thereby establishing the model. In the present method, the attribute of the breast lesion is selected from the group consisting of location, margin, calcification, lump, mass, shape, size, status of the breast lesion, and a combination thereof.

According to some embodiments of the present disclosure, in step (c) of the present method, upon being detected, the attribute on each of the processed images of step (b) is framed to produce a framed image.

In some alternative or optional embodiments, the present method further comprises mask filtering the framed image and the segmented image of step (c) to eliminate any mistaken attribute detected in step (c). In this scenario, the extracted sub-image of step (c) is produced by cropping the framed image.

In some alternative or optional embodiments, after step (d) or step (e) of the present method, it further comprises updating the segmented image of step (c) with the aid of the segmented sub-image of step (d) and the framed image.

According to some embodiments of the present disclosure, in step (c) of the present method, the attribute on each of the processed images of step (b) is detected by use of an object detection algorithm.

According to some embodiments of the present disclosure, in step (c) of the present method, each of the processed images is segmented by use of a U-net architecture.

According to one embodiment of the present disclosure, the subject is a human.

In another aspect, the present disclosure pertains to a method for treating a breast cancer via determining a breast lesion in a subject. The method comprises: (a) obtaining a mammographic image of the breast from the subject, in which the mammographic image comprises an attribute of the breast lesion selected from the group consisting of location, margin, calcification, lump, mass, shape, size, status of the breast lesion, and a combination thereof; (b) producing a processed image via subjecting the mammographic image to image treatments selected from the group consisting of image cropping, image denoising, image flipping, histogram equalization, image padding, and a combination thereof; (c) segmenting the processed image of step (b) to produce a segmented image, and/or detecting the attribute on the processed image of step (b), thereby producing an extracted sub-image thereof; (d) segmenting the extracted sub-image of step (c) to produce a segmented sub-image; (e) combining the extracted sub-image of step (c) and the segmented sub-image of step (d), thereby producing a text image exhibiting the attribute for the mammographic image; (f) determining the breast lesion of the subject by processing the text image of step (e) within the model established by the aforementioned method; and (g) providing an anti-cancer treatment to the subject based on the breast lesion determined in step (f).

According to one embodiment of the present disclosure, in step (c) of the present method, the attribute on the processed images of step (b) is framed to produce a framed image upon being detected.

In some alternative or optional embodiments, the present method further comprises mask filtering the framed image and the segmented image of step (c) to eliminate any mistaken attribute detected in step (c). In still some alternative or optional embodiments, the present method further comprises cropping the framed image to produce the extracted sub-image of step (c).

Alternatively or optionally, after step (d) or step (e) of the present method, it further comprises updating the segmented image of step (c) with the aid of the segmented sub-image of step (d) and the framed image.

According to some embodiments of the present disclosure, the attribute on the processed image of step (b) is detected by use of an object detection algorithm.

According to some embodiments of the present disclosure, in step (c) of the present method, the processed image is segmented by performing a U-net architecture.

According to some embodiments of the present disclosure, in step (g) of the present method, the anti-cancer treatment is selected from the group consisting of a surgery, a radiofrequency ablation, a systemic chemotherapy, a transarterial chemoembolization (TACE), an immunotherapy, a targeted drug therapy, a hormone therapy, and a combination thereof.

According to one embodiment of the present disclosure, the subject is a human.

By virtue of the above configuration, the model established by the method of the present invention can identify the attributes of breast lesions in mammographic images and determine the category of breast lesions in a rapid manner, thereby improving the efficiency and accuracy in diagnosis of breast cancers.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a flow chart of a method 10 according to one embodiment of the present disclosure; and

FIG. 2 is a flow chart of a method 20 according to another embodiment of the present disclosure.

In accordance with common practice, the various described features/elements are not drawn to scale but instead are drawn to best illustrate specific features/elements relevant to the present invention. Also, like reference numerals and designations in the various drawings are used to indicate like elements/parts.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. Definition

For convenience, certain terms employed in the specification, examples and appended claims are collected here. Unless defined otherwise, all technical and scientific terms used herein have the same meaning as commonly understood by one of the ordinary skill in the art to which this invention belongs.

The singular forms “a”, “and”, and “the” are used herein to include plural referents unless the context clearly dictates otherwise.

The term “breast lesion” as used herein is intended to encompass any abnormal tissue detected through breast mammography, including both benign (non-cancerous) and malignant (cancerous) conditions such as cysts, fibroadenomas, and malignant tumors.

The term “attribute” as used herein refers to the characteristics or features of the breast lesion detected or captured in mammographic images, which are crucial for determining the nature of the breast lesion. Examples of attributes commonly used in mammography to describe breast lesions include, but are not limited to, size, shape, margins, density, calcifications, masses, vascularity, location, and status. The term “status” as used herein refers to whether the breast lesion is cancerous (or malignant) or non-cancerous (or benign).

The term “image treatments(s)” as used herein is intended to encompass all processing procedures applied to raw images through digital computer algorithms to enhance, transform, or extract information from the images. According to the present disclosure, the terms “image treatment(s)” and “image processing” are used interchangeably as they bear the same meaning.

The term “combined image” as used herein refers to an image an image composed of a segmented image and a cropped image derived from a raw image for object detection (i.e., lesion detection in the present application). This combined image is then used for training a machine learning algorithm, thereby building the present model. According to the present disclosure, the combined images used for training a machine learning model serve as “reference images”, whereas the combined image that obtained from a subject for identifying his/her breast lesion serves as a “test image.”

As used herein, the term “treat,” “treating” and “treatment” are interchangeable, and encompasses partially or completely preventing, ameliorating, mitigating and/or managing a symptom, a secondary disorder or a condition associated with breast cancer.

### 2. Description of the Invention

Clinically, the interpretation of mammographic images relies on experienced professionals. To enhance the accuracy of assessment, this invention aims to provide a method for establishing a model to identify breast lesions in mammographic images. Also herein is a method for determining and treating breast cancer with the aid of the established model.

### 2.1 Method for Building a Model for Breast Lesion Determination

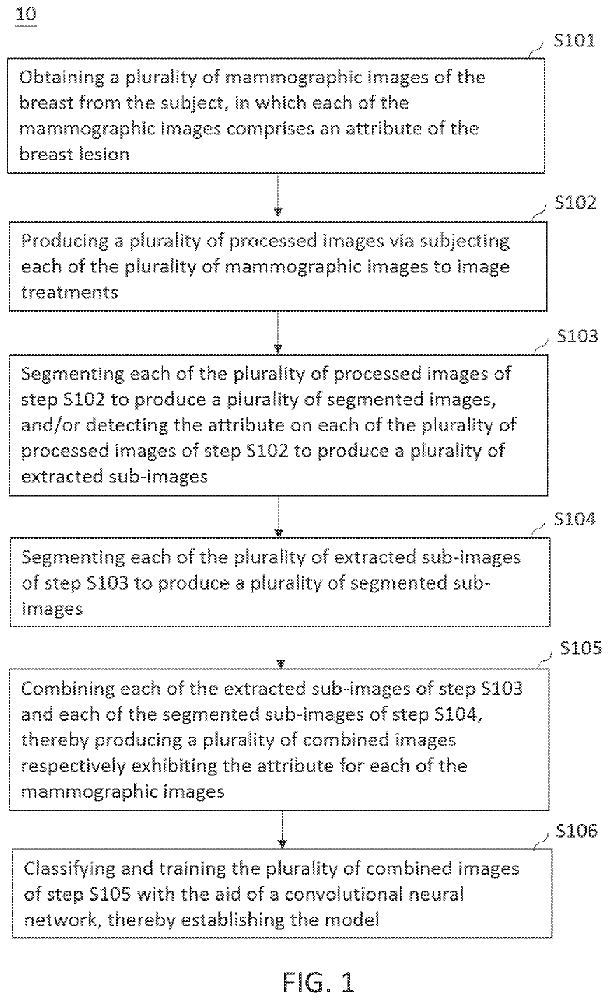

The first aspect of the present disclosure is directed to a method for building a model for determining breast lesion via mammographic images from a subject. Reference is made to FIG. 1.

FIG. 1 is a flow chart of a method 10 implemented on a computer or a processor according to one embodiment of the present disclosure. The method 10 comprises the following steps, which are respectively indicated by reference numbers S101 to S106 in FIG. 1,
S101: obtaining a plurality of mammographic images of the breast from the subject, in which each of the mammographic images comprises an attribute of the breast lesion;
S102: producing a plurality of processed images via subjecting each of the plurality of mammographic images to image treatments;
S103: segmenting each of the plurality of processed images of step S102 to produce a plurality of segmented images, and/or detecting the attribute on each of the plurality of processed images of step S102 to produce a plurality of extracted sub-images;
S104: segmenting each of the plurality of extracted sub-images of step S103 to produce a plurality of segmented sub-images;
S105: combining each of the extracted sub-images of step S103 and each of the segmented sub-images of step S104, thereby producing a plurality of combined images respectively exhibiting the attribute for each of the mammographic images; and
S106: classifying and training the plurality of combined images of step S105 with the aid of a convolutional neural network, thereby establishing the model.

According to embodiments of the present disclosure, the mammographic images are low dose X-ray images of breasts obtained from a healthy or a diseased subject, preferably from a healthy or a diseased human. In order to build and train the model, multiple mammographic images derived from subjects and independently contain known attributes of breast lesions are used in the present training method 10. Practically, the mammographic images of breasts may be collected from existing databases of medical centers or competent authority of health, whether publicly accessible or not (S101). According to embodiments of the present disclosure, the attribute of the breast lesion comprises location, margin, calcification, lump, mass, shape, size, and status of the breast lesion, and/or a combination thereof. In practice, the diagnostic information (e.g., categories 0 to 6 of BI-RADS) corresponding to each mammographic image and subject may also be collected for reference. Then, the mammographic images are automatically forwarded to a device and/or system (e.g., a computer or a processor) having instructions embedded thereon for executing the subsequent steps (S102 to S106).

According to embodiments of the present disclosure, in step S102, the forwarded mammographic images are subjected to image processing to transform them into regularized and standardized images. In one working example, the mammographic images are processed sequentially with image processing treatments of image cropping, image denoising, image flipping, histogram equalization, and image padding, thereby producing multiple processed images. Each of image treatments can be performed by use of algorithms well known in the art, so as to standardize and regularize raw mammographic images for subsequent usage. Specifically, the image cropping is designed to eliminate the edges of the input image, which may be affected by noise from mammography. Examples of image cropping software include but are limited to, Adobe Photoshop, GIMP (GNU Image Manipulation Program), Microsoft Paint, IrfanView, Photoscape, Snagit, Pixlr, Fotor, Canva, and Paint.NET. The image denoising treatment as used in the present method involves the use of various filters to achieve noise reduction. Practical tools suitable for image denoising include Adobe Photoshop, Topaz DeNoise AI, DxO PhotoLab, Noiseware, Neat Image, and Dfine; yet not limited hereto. The main purpose of image flipping is to flip each mammographic image to the same orientation, reducing the calculation time and improving the processing speed required in subsequent steps (e.g., model training). Those skilled in the art can use well known tools including Adobe Photoshop, GIMP (GNU Image Manipulation Program), Microsoft Paint, and the like to achieve image flipping. Histogram equalization enhances the contrast of the mammographic image by redistributing the intensity values across its histogram, resulting in the processed image with improved visibility of details and enhanced visual quality. Examples of tools capable of performing histogram equalization include but are not limited to, MATLAB, OpenCV, ImageJ, Scikit-image, Fiji, and Adobe Photoshop. Once a variation in size is observed among the raw mammographic images, image padding is performed to standardize the sizes by adding extra pixels to the borders of the images. Examples of well-known image padding tools suitable for use in the present method include but are not limited to, OpenCV, NumPy, Python Imaging Library (PIL), TensorFlow, and the like. After aforementioned treatments, the processed images are then used for subsequent model training and learning algorithms, so as to ensure consistency and standardization by eliminating the impact of irregular size, edges, or noise in raw mammographic images.

In step S103, each of the processed images may be subjected to (I) image segmentation, and/or (II) object detection described below.

### (I) Image Segmentation

In this process, each of the processed images are segmented to produce a plurality of segmented images. According to embodiments of the present disclosure, various convolutional networks architectures (e.g., U-net architecture) may be used to identify and isolate the areas corresponding to breast lesions on the processed images. In some embodiments of the present disclosure, the U-net architecture, examples of which include, but are not limiting to, Swin-Unet, TransUnet, and a combination thereof, is used for segmenting the processed images. Alternatively or optionally, adaptive modification may be made to the U-net architecture to enhance segmentation efficiency. In one working example, when the U-net architecture is applied for image segmentation, Symlet wavelet filtering is combined with max pooling or average pooling before downsampling. The combination of wavelet filtering with pooling enables the U-net architecture to retain important frequency information while reducing the spatial resolution.

### (II) Object Detection

To the purpose of object detection, each of the processed images is subjected to object detection, in which the attribute(s) on each of the processed images is/are detected, leading to the production of a plurality of extracted sub-images. According to embodiments of the present disclosure, the object detection is achieved by performing various object detection algorithms known in the art. Examples of object detection algorisms suitable for use in the present method include, but are not limited to, two-stage detector2 00: convolutional neural networks (R-CNN, Region-Based Convolutional Neural Networks), Fast R-CNN, Faster RCNN, RFCN, mask RCNN; and one-stage detector, e.g., YOLO (You Only Look Once), and SSD (single shot detector). In working examples, object detection is achieved by use of both YOLOv7 and EfficientDet algorisms. Accordingly, the attributes of the breast lesions in the mammographic images including location, margin, calcification, lump, mass, shape, size, and the like can be comprehensively detected.

In preferred embodiments, the attribute detected in (II) object detection process is framed, thereby producing a framed image. As a result, the visual representation of the framed image will include the lesions that have been detected, outlined within bounding boxes.

According to embodiments of the present disclosure, either (I) or (II) processes described above is chosen for executing step (c). Alternatively, both (I) and (II) processes are proceeded simultaneously.

Additionally, in preferred embodiments, mask filtering is applied to segmented and framed images that are obtained from the aforementioned (I) and/or (II) processes, so as to eliminate any mistaken attribute detected in (I) and/or (II) processes. As a result, the probability of miscalculation can be significantly reduced.

Subsequently, each of the framed images is cropped to generate an extracted sub-image, facilitating the acquisition of more detailed information about the attributes. After assembling a collection of multiple extracted sub-images, they are sent to the next step, which involves further segmentation of the multiple extracted sub-images (Step S104).

In step S104, segmentation of each extracted sub-image is achieved by use of the U-net architecture as previously described in step S103, resulting in the generation of a plurality of segmented sub-images.

Once the segmented sub-images are produced, each of the extracted sub-images from step S103 is combined and overlaid with its corresponding segmented sub-image produced in step S104 (step S105). Consequently, each combined image exhibits the respective attributes for each of the mammographic images obtained in step S101. This step allows for producing clearer information about the lesion's location and boundaries, thereby enhancing the resolution for improved accuracy in subsequent classification and learning processes.

In preferred embodiments of the present disclosure, after step S104 or S105, the segmented image of step S103 can be updated with the aid of the segmented sub-image and the framed image. Specifically, the segmented sub-image and the framed image are overlaid and fed into the plurality of processed images, thus adjusting the segmentation and the mask filtering processes set forth above, thereby generates updated segmented images. This refinement enables the images used to construct the model of the present disclosure to have clearer information about breast lesions.

Finally, in step S106, a convolutional neural network, well-established in the art, is utilized to classify and train the plurality of combined images produced in step S105, thereby establishing the present model. Examples of convolutional neural network (CNN) suitable for use in the present method include but are not limited to, LeNet-5, AlexNet, VGGNet (VGG16 and VGG19), GoogLeNet (Inception), ResNet (Residual Network), MobileNet, YOLO, Faster R-CNN, U-net, EfficientNet, and a combination thereof. In working examples, the classification and training are performed by use of EfficientNet-V2. According to embodiments of the present disclosure, during the training step, multiple classifiers based on various attributes (including location, margin, calcification, lump, mass, shape, size, etc.) are established, thereby enhances learning efficiency.

According to alternative embodiments of the present disclosure, the combined image is classified by use of a complex-sparse matrix factorization method and the convolutional neural network (e.g., EfficientNet-V2). Specifically, the complex-sparse matrix factorization method is applied to the attributes in the combined images to yield one category score, while the CNN model is applied in similar manner to produce another category score. The classification of the attributes in the combined images is determined by the summation of these two category scores. In some working embodiments, the outcome of the complex-sparse matrix factorization method is utilized to infer the similarity between features of trained images using the k-nearest neighbors (k-NN) algorithm, subsequently transforming them into corresponding category scores.

In practical implementation according to embodiments of the present disclosure, the method 10 is executed through a processor programmed with instructions and/or a system that includes the processor for carrying out the method 10. Specifically, the processor is configured to perform image processing, segmentation, object detection, image classification, and training for the establishment of the present model for breast lesion determination. Accordingly, in preferred embodiment, the present method 10 is implemented on the processor for building the model for breast lesion determination.

By performing the afore-mentioned steps S101 to S106, a model well-trained for determining a breast lesion is established. The established model of the present disclosure can effectively discriminate breast lesions in mammographic images of a human and automatically interpret the BI-RADS categories.

### 2.2 Methods for Determining and Treating Breast Cancers

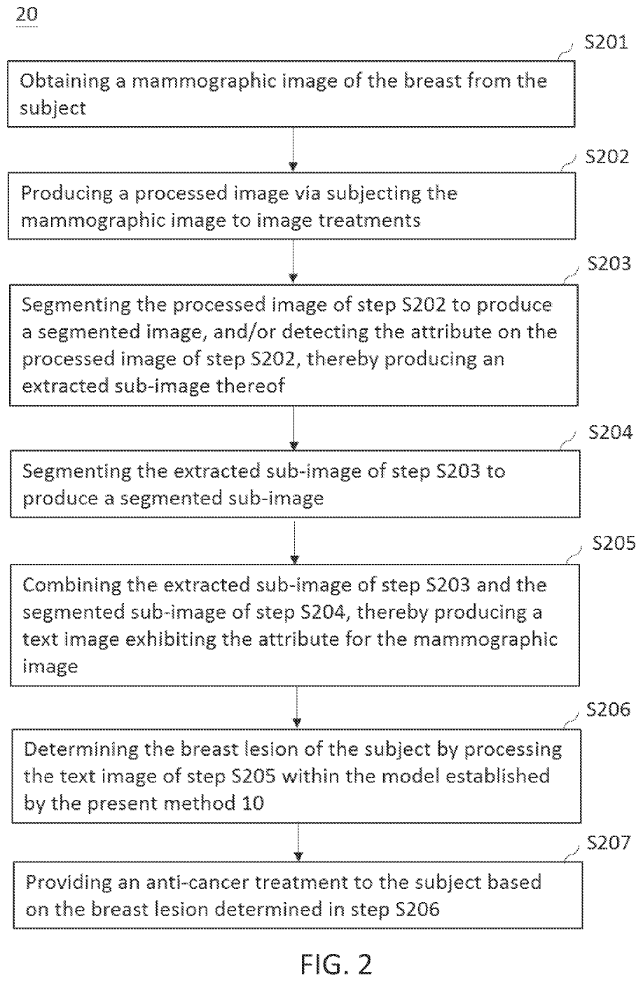

The present disclosure also aims at providing diagnosis and treatment to a subject afflicted with, or suspected of developing a breast cancer. To this purpose, the method and model described in section 2.1 of this paper may be utilized to assist physicians with precise determination of breast lesions on mammographic images. The present disclosure thus encompasses another aspect that is directed to a method for determining and treating a breast cancer in a subject. References are made to FIG. 2.

FIG. 2 depicts a flow chart of a method 20 for determining a breast cancer via determining a breast lesion in a subject, who is having or suspected of having a breast cancer.

The method 20 includes the following steps (see the reference numbers S201 to S207 indicated in FIG. 2),
S201: obtaining a mammographic image of the breast from the subject;
S202: producing a processed image via subjecting the mammographic image to image treatments selected from the group consisting of image cropping, image denoising, image flipping, histogram equalization, image padding, and a combination thereof;
S203: segmenting the processed image of step S202 to produce a segmented image, and/or detecting the attribute on the processed image of step S202, thereby producing an extracted sub-image thereof;
S204: segmenting the extracted sub-image of step S203 to produce a segmented sub-image;
S205: combining the extracted sub-image of step S203 and the segmented sub-image of step S204, thereby producing a text image exhibiting the attribute for the mammographic image;
S206: determining the breast lesion of the subject by processing the text image of step S205 within the model established by the present method 10; and
S207: providing an anti-cancer treatment to the subject based on the breast lesion determined in step S206.

The present method 20 begins by obtaining a mammographic image of the breast from the subject, which may be a mammal, for example, a human, a mouse, a rat, a hamster, a guinea pig, a rabbit, a dog, a cat, a cow, a goat, a sheep, a monkey, or a horse. Preferably the subject is a human. Suitable tool and/or procedures may be performed to obtain the mammographic image. In one working example, the mammographic image is captured and collected by a mammography machine using a low dose of X radiation (step S201). Typically, the thus collected mammographic image comprises an attribute of the breast lesion.

Then, the mammographic image can be processed to produce a processed image (step S202), which is further subjected to segmentation and object detection described in steps S203 to S204. Like steps S102 to S104 of the method 10, the strategies utilized in steps S202 to S204 can be achieved by use of algorithms well-known in the art. For example, the image treatments of step S202 can be achieved by using image processing software such as Adobe Photoshop, MATLAB, OpenCV, Python Imaging Library (PIL), and the like; yet not limited thereto. As for segmentation and object detection in step S203 and S204, they can be achieved by the same algorisms (e.g., U-net architecture and convolutional neural networks) and criteria as those indicated in step S103 and S104 of the method 10. For the sake of brevity, steps S202 to S204 are not reiterated herein.

Proceed to steps S205 and S206, the test image exhibiting the attribute for the mammographic image of the subject is produced by combining the extracted sub-image of step S203 and the segmented sub-image of step S204 and then subjected to analysis via the model established by the present method 10, in which the attributes of the test image are compared with those in reference images constructed in the model, so as to determine the breast lesion thereof.

According to embodiments of the present disclosure, the attributes of the breast lesion include but are not limited to location, margin, calcification, lump, mass, shape, size, status of the breast lesion, and a combination thereof. After inputting the test image into the present model, the k-nearest neighbors (k-NN) algorithm is executed. Based on the learned classifiers, detailed information about the lesion attributes within the test image can be determined. Subsequently, in accordance with this information and with the assistance of BI-RADS, clinical practitioners can assess the risk level of abnormalities. When the score falls within the categories of 4-6, further examinations are required, and/or a malignant lesion is determined.

Once the malignant lesion of the breasts is determined and confirmed, proper anti-cancer treatment(s) may be timely administered to the subject. Examples of anti-cancer treatment suitable for use in the present method (i.e., for administering to a subject whose breast lesion is determined malignant) include, but are not limited to, surgery, radiofrequency ablation, systemic chemotherapy, transarterial chemoembolization (TACE), immunotherapy, targeted drug therapy, hormone therapy, and a combination thereof. Any clinical artisans may choose a suitable treatment for use in the present method based on factors such as the particular condition being treated, the severity of the condition, the individual patient parameters (including age, physical condition, size, gender, and weight), the duration of the treatment, the nature of concurrent therapy (if any), the specific route of administration and like factors within the knowledge and expertise of the health practitioner.

By virtue of the above features, the present method can provide precise determination and identification of breast lesions mainly based on mammographic images in a rapid, excise manner, thereby improving the accuracy and efficiency of breast cancer diagnosis and allowing the identified patients to be treated properly.

### EXAMPLES

### Materials and Methods

### Data Collection

A total of 52,770 mammographic images of breast lesions were obtained from the Department of Breast surgery in Mackay Memorial Hospital (Taipei City) and used for constructing a model of image recognition and verification.

### Image Processing

Every mammographic image obtained from the database was subjected to treatments of image cropping, image denoising, image flipping, histogram equalization, and image padding, thereby rectifying into a regularized pixel size of 1,280×1,280 for further model construction with the aid of EfficientDet, YOLOv7, Swin-Unet, TransUnet, and EfficientNet-V2.

### Example 1 Constructing Image Recognition Model of the Present Disclosure

This experiment aimed at providing a machine learning model trained for breast lesion recognition. To this purpose, one model capable of recognizing attributes of the breast lesion was established in accordance with the procedures outlined in section 2.1 and the “materials and methods” section. Specifically, a total of 42,200 mammographic images including various attributes were used.

### Example 2 Evaluation of the Present Model

Next, the image recognition efficiency of the trained model and method for breast lesion determination of Example 1 was verified. To this purpose, more than 10,000 candidate mammographic images were processed and input into the present model.

It was found that the F1 score, precision, and recall of the present model are respectively 0.91, 0.86, and 0.95, indicating high accuracy in the assessment and determination of breast lesions.

By using the present method and system, the mammograms obtained from patients can be automatically interpreted and identified, thereby improving the efficiency and accuracy of breast cancer diagnosis.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for building a model for determining a breast lesion in a subject, comprising:
(a) obtaining a plurality of mammographic images of the breast from the subject, in which each of the mammographic images comprises an attribute of the breast lesion selected from the group consisting of location, margin, calcification, lump, mass, shape, size, status of the breast lesion, and a combination thereof;
(b) producing a plurality of processed images via subjecting each of the plurality of mammographic images to image treatments selected from the group consisting of image cropping, image denoising, image flipping, histogram equalization, image padding, and a combination thereof;
(c) segmenting each of the plurality of processed images of step (b) to produce a plurality of segmented images, and/or detecting the attribute on each of the plurality of processed images of step (b) to produce a plurality of extracted sub-images;
(d) segmenting each of the plurality of extracted sub-images of step (c) to produce a plurality of segmented sub-images;
(e) combining each of the extracted sub-images of step (c) and each of the segmented sub-images of step (d), thereby producing a plurality of combined images respectively exhibiting the attribute for each of the mammographic images; and
(f) classifying and training the plurality of combined images of step (e) with the aid of a convolutional neural network, thereby establishing the model.

2. The method of claim 1, wherein in step (c), upon being detected, the attribute on each of the processed images of step (b) is framed to produce a framed image.

3. The method of claim 2, further comprising mask filtering the framed image and the segmented image of step (c) to eliminate any mistaken attribute detected in step (c).

4. The method of claim 3, further comprising cropping the framed image to produce the extracted sub-image of step (c).

5. The method of claim 4, further comprising, after step (d) or step (e), updating the segmented image of step (c) with the aid of the segmented sub-image of step (d) and the framed image.

6. The method of claim 1, wherein in step (c), the attribute on each of the processed images of step (b) is detected by use of an object detection algorithm.

7. The method of claim 1, wherein in step (c), each of the processed images is segmented by use of a U-net architecture.

8. The method of claim 1, wherein the subject is a human.

9. A method for treating a breast cancer via determining a breast lesion in a subject, comprising:
(a) obtaining a mammographic image of the breast from the subject, in which the mammographic image comprises an attribute of the breast lesion selected from the group consisting of location, margin, calcification, lump, mass, shape, size, status of the breast lesion, and a combination thereof;
(b) producing a processed image via subjecting the mammographic image to image treatments selected from the group consisting of image cropping, image denoising, image flipping, histogram equalization, image padding, and a combination thereof;
(c) segmenting the processed image of step (b) to produce a segmented image, and/or detecting the attribute on the processed image of step (b), thereby producing an extracted sub-image thereof;
(d) segmenting the extracted sub-image of step (c) to produce a segmented sub-image;
(e) combining the extracted sub-image of step (c) and the segmented sub-image of step (d), thereby producing a text image exhibiting the attribute for the mammographic image;
(f) determining the breast lesion of the subject by processing the text image of step (e) within the model established by the method of claim 1; and
(g) providing an anti-cancer treatment to the subject based on the breast lesion determined in step (f).

10. The method of claim 9, wherein in step (c), upon being detected, the attribute on the processed images of step (b) is framed to produce a framed image.

11. The method of claim 10, further comprising mask filtering the framed image and the segmented image of step (c) to eliminate any mistaken attribute detected in step (c).

12. The method of claim 11, further comprising cropping the framed image to produce the extracted sub-image of step (c).

13. The method of claim 12, further comprising, after step (d) or step (e), updating the segmented image of step (c) with the aid of the segmented sub-image of step (d) and the framed image.

14. The method of claim 9, wherein in step (c), the attribute on the processed image of step (b) is detected by use of an object detection algorithm.

15. The method of claim 9, wherein in step (c), the processed image is segmented by performing a U-net architecture.

16. The method of claim 9, wherein in step (g), the anti-cancer treatment is selected from the group consisting of a surgery, a radiofrequency ablation, a systemic chemotherapy, a transarterial chemoembolization (TACE), an immunotherapy, a targeted drug therapy, a hormone therapy, and a combination thereof.

17. The method of claim 9, wherein the subject is a human.
